(19) United States
(12) Patent Application Publication
(10) Pub. No.: US 2020/0337536 A1
(43) Pub. Date: Oct. 29, 2020

(54) IMAGING DEVICE FOR USE WITH SURGICAL INSTRUMENT

(71) Applicants: Septimiu SALCUDEAN, Vancouver (CA); Omid MOHARERI, San Francisco, CA (US); Prateek MATHUR, Toronto (CA); Apeksha AVINASH, Chennai (IN); Alaa Eldin ABDELAAL, Vancouver (CA)

(72) Inventors: Septimiu SALCUDEAN, Vancouver (CA); Omid MOHARERI, San Francisco, CA (US); Prateek MATHUR, Toronto (CA); Apeksha AVINASH, Chennai (IN); Alaa Eldin ABDELAAL, Vancouver (CA)

(21) Appl. No.: 16/862,371

(22) Filed: Apr. 29, 2020

Related U.S. Application Data
(60) Provisional application No. 62/839,963, filed on Apr. 29, 2019.

(51) Int. Cl. A61B 1/00; A61B 1/313; A61B 1/12; A61B 1/05; A61B 34/37

(52) U.S. Cl. CPC A61B 1/00193; A61B 1/3132; A61B 1/00066; A61B 2034/301; A61B 1/00177; A61B 1/05; A61B 34/37; A61B 1/126

(57) ABSTRACT

A device for imaging in a body cavity (e.g. for laparoscopic surgery) has a body dimensioned for insertion by way of a trocar. The body carries plural side-facing cameras. A gripping interface on the body is configured to allow the device to be grasped by a surgical implement and positioned to view a location relevant to a surgical procedure. Some embodiments provide adjustable baselines for stereoscopic viewing. An irrigation system may be provided for cleaning lenses of the cameras while the imaging device remains in a body cavity.

### CROSS-REFERENCE TO RELATED APPLICATIONS

This application claims the benefit under 35 U.S.C. § 119 of U.S. Provisional Patent Application No. 62/839,963 filed 29 Apr. 2019 and entitled IMAGING DEVICE FOR USE WITH SURGICAL INSTRUMENT which is hereby incorporated herein by reference for all purposes.

### FIELD OF THE INVENTION

The present technology relates to the field of medical imaging. The technology has example application in minimally invasive surgery. Some embodiments provide imaging devices and methods useful in teleoperated robotic surgery.

### BACKGROUND

Minimally invasive surgery is carried out with minimally invasive instruments maneuvered through small incisions into the patient's insufflated body. An imaging device such as an endoscope is first inserted into the body to provide the surgeon with images from the surgical field. Using these images, the surgeon manipulates the surgical instruments and performs the surgery. An increasingly popular form of minimally invasive surgery is robot-assisted surgery in which robotic arms of a patient side robot are inserted into incisions and these arms are teleoperatively controlled by a surgeon sitting at an operating console which includes master controllers for the robotic arms. the console may be anywhere in the world (but is often a few steps from the operating field).

Movements made by the surgeon on the master controllers are reflected in motions of the robotic arms of the patient-side robot. The robotic arms typically include wristed instruments which can be maneuvered with six degrees of freedom (DOF) inside the patient's body. Robot-assisted surgery brings with it improved precision, increased accuracy, tremor reduction, and improved dexterity over traditional minimally invasive laparoscopic surgery, which typically does not have wristed instruments and provides for only four DOF.

Teleoperated robotic surgical systems include a patient-side robotic arm that holds an endoscopic camera. The endoscopic camera is typically the sole source of images of the surgical field. However, the point of insertion of the endoscope and the kinematics of the robotic arm that holds the endoscope limit the areas that can be imaged. The limit field of view provided by syc endoscopic cameras has been a problem since the early days of robot-assisted surgery.

Okada et al., U.S. Pat. No. 5,653,677A titled ‘Electronic endoscope apparatus with imaging unit separable therefrom’ proposes an endoscope that includes a detachable imaging device at its tip. The imaging device can be forcibly detached by means of air or water and can be propelled to a region of interest. The imaging device may have self-propelling capabilities but otherwise its movement cannot be controlled.

Nishiyama et al., U.S. Pat. No. 8,328,712B2 titled ‘Image processing system, external device and image processing method’, describes a body-insertable capsule-like imaging device that can have a number of imaging units that transmit images wirelessly to an external monitor. Each imaging unit includes a Charge Coupled Device (CCD) array, sources of illumination (LED), an objective lens system, and drive circuitry for both the CCD array and the LEDs. Each imaging unit also transmits an identification number to the external device. This external device has a selecting unit that allows the operator to choose which image data is to be displayed on the monitor.

Nishino, U.S. Pat. No. 8,562,515B2 titled ‘Capsule endoscope, capsule endoscopic system, and endoscope control method’, describes a capsule endoscope system with multiple imaging units. The capsule includes a direction detector (an acceleration sensor and an integrator) to detect the direction of the imagining units inside the body.

Shigemori, U.S. Pat. No. 8,382,658B2 titled ‘Capsule endoscope system’ describes inserting multiple such capsule endoscopes to provide multiple surgical views. An external monitor indicates which device is capturing the images being displayed at any point of time.

Asada et al., U.S. Pat. No. 8,348,828B2 titled ‘Medical apparatus and operation method for introducing medical apparatus into body’, describes an imaging device that is introduced inside the body cavity and then attached to the abdominal wall with the help of a needle.

The imaging device has a grasping portion on one side. An instrument such as the forceps is used to grasp the imaging device through this portion to insert it through a trocar and also to remove it from the body cavity. The imaging device has a cap with a needle that is used to puncture the abdominal wall. The imaging device contains a lens system to provide wide-angle Field of Views (FOVs), sources of illumination (LEDs), a transmitter, battery, and may also have zoom capabilities. It also contains electromagnetic coils to facilitate the flow of current from an externally connected power supply. However, once fixed to the abdominal wall, the imaging device cannot be easily moved to new positions.

Whitman, U.S. Pat. No. 7,751,870B2 titled ‘Surgical imaging device’, describes a surgical imaging device that can be inserted into a body cavity. The device has a number of bendable prongs with imaging units on each of these prongs to provide different views. In an initial position of the device all of the prongs are parallel to each other for easy insertion through a surgical trocar. Once inserted, the operator uses a control lever to radially separate the prongs to any desired position. Controls can be transmitted either through a wired connection or wirelessly.

Adnair, U.S. Pat. No. 6,086,528A titled ‘Surgical devices with removable imaging capability and methods of employing same’, describes an imaging device that can be attached to a surgical instrument and a modified surgical instrument that includes a tube through which a microendoscope can be passed. The microendoscope contains an image sensor, light source, and a tube that carries fibre optic cables and cleaning mechanisms.

Whitman et al., U.S. Pat. No. 8,771,169B2 titled ‘Imaging system for a surgical device’, describes a pivotable camera assembly that can be attached to the shaft of a surgical instrument. The camera assembly may include at least one camera and at least one light source for illumination, and is moveable between two positions. The surgical device may also include a control mechanism to control the movement of the camera assembly between the two positions. The camera projects outward from the shaft at a certain distance from the distal end of the instrument. There may be two openings on radially opposite ends of the shaft through which the camera assembly can emerge. The movement can be either manual or automatic.

Whitman et al., U.S. Pat. No. 8,262,560B2 titled ‘Imaging device for use with a surgical device’, describes an imaging device that can be detachably coupled to a circular or linear stapler. The imaging device includes a lens system, light source, image sensor, and a cleaning arrangement. The imaging device can receive control signals for controlling zoom, illumination and the flow rate of water/air for cleaning. The imaging device is connected to an external electro-mechanical driver device for transmitting images (either through wired connections or wirelessly) and for control signals.

Stelzer et al., U.S. Pat. No. 6,309,345B1 titled ‘Minimally invasive surgery device’, describes a surgical device with a rotatable node at its distal end. A surgical instrument (including an endoscope) can be attached to this node and can be rotated in the X and Y directions, and can be displaced along the Z direction (insertion and retraction). Specially designed instruments are to be used with this device that could also include at least two cameras to provide stereoscopic vision. Illumination is provided by two fibre optic sources. A single such surgical device can have multiple nodes that can each hold a surgical instrument. The shaft of the device can be bendably controlled.

### BRIEF SUMMARY OF THE INVENTION

This invention has several different aspects these include without limitation:
devices for imaging inside a body cavity;
stereoscopic endoscopic imaging devices;
methods for imaging inside a body cavity;
methods for controlling robotic surgical tools;
methods and apparatus for setting coordinate frames for controlling surgical tools; and
robotic surgery systems,

One aspect of the invention provides imaging devices useful for viewing images of the inside of the body cavity in minimally invasive surgery. The imaging devices include interfaces that facilitate grasping the imaging devices with a surgical instrument. The surgical instrument can then be manipulated to hold the imaging device in a desired position. The interfaces are configured so that a geometry of the imaging devices relative to a surgical tool grasping the imaging device is known and fixed.

In some embodiments the imaging device has a cylindrical configuration, the grasping interface may be located at or close to a proximal end of the imaging device. A suitable surgical instrument with jaws is operated to mate with the grasping interface to pick up and hold the imaging device. The grasping interface is designed such that the grasping is repeatable and the instrument maintains a secure hold on the imaging device when its jaws are closed. Some embodiments provide a grasping interface that is removably attachable from the imaging device. Different grasping interfaces may be attached to the same imaging device to facilitate repeatable grasping by different surgical implements.

In some embodiments the imaging device comprises at least two imaging units (cameras) present on a lateral surface along the longitudinal axis of the imaging device. Images from the at least two imaging units may provide stereoscopic views of a surgical scene. In some embodiments at least one source of illumination is provided between or near the imaging units.

In some embodiments a single imaging unit present on a distal end of the imaging device. The single imaging unit may provide a view directed along an axis of the imaging device. One or more sources of illumination may be provided on the distal surface.

In some embodiments, images captured by all imaging units present on the device are transmitted to an external display for viewing.

In some embodiments, the imaging device can be tracked in physical space using robotic forward kinematics. Thus the location and orientation of the imaging device within the body cavity can be determined at any time.

Another aspect of the invention provides an imaging device. The imaging device comprises an elongated body and a plurality of side-looking cameras each comprising an imaging sensor and a lens system. The cameras are spaced apart from one another in a direction along the body and facing sideways. A grasping interface is provided on the body. The grasping interface is configured to be grasped by jaws of a surgical instrument. The body may have a cylindrical outer surface. The grasping interface may be entirely inside a cylindrical envelope coinciding with the cylindrical outer surface of the body.

In some embodiments the grasping interface comprises first and second opposed grooves extending transversely to the body. The first and second opposed grooves comprise inclined sidewalls such that a base surface of the first groove is narrower than an opening of the first groove and a base surface of the second groove is narrower than an opening of the second groove. The base surface of the first groove may be inclined at an acute angle relative to the base surface of the second groove.

In some embodiments the grasping interface comprises first and second projections extending generally perpendicularly from the base surfaces of the first and second grooves respectively. These projections may engage slots, apertures or recesses in jaws of an implement used to grasp the grasping interface.

In some embodiments the first and second projections are spaced apart from the sidewalls of the first and second grooves respectively. For example one or both of the first and second projections may be centered between the sidewalls.

In some embodiments the grasping interface comprises a plurality of sets of base surfaces, each of the sets of base surfaces comprising a first base surface inclined at an acute angle to a second base surface. Each of the pairs of base surfaces may be symmetrical about a plane that includes a longitudinal axis of the body. The pairs of base surfaces may be equally angularly spaced apart around a circumference of the body. In some embodiments first and second projections extend generally perpendicularly from the first and second base surfaces of each of the pairs of base surfaces. The grasping interface may, for example, comprise three to five pairs of the base surfaces.

In some embodiments the body is cylindrical and has an outside diameter of less than 11 mm. For example, the body may have a diameter of about 10.5 mm so that it can be slid through a passage having a diameter of 11 mm or the body may have a diameter of about 5.5 mm so that it can be slid through a passage having a diameter of 6 mm.

Some embodiments provide one or more additional cameras on a distal end of the body.

In some embodiments an umbilical is connected to a proximal end of the body. The umbilical may carry conductors for power, control signals, and/or fluids. For example, the umbilical may comprise at least one lumen in fluid communication with one or more orifices, the one or more orifices located adjacent to and oriented to eject fluid onto an outer surface of the lens system of at least one of the cameras.

In some embodiments a baseline distance BL between optical axes of first and second ones of the plurality of side-looking cameras is adjustable. For example, the body may comprise a first part carrying the first one of the plurality of side-looking cameras and a second part carrying the second one of the plurality of side-looking cameras and the first part may be movable relative to the second part (e.g. by being telescopically received in the second part). In some embodiments a power actuator is coupled to drive the first one of the plurality of side-looking cameras toward or away from the second one of the plurality of side-looking cameras.

In some embodiments the grasping interface is provided by a first grasping interface that is detachably coupled to the body and the imaging device comprises a second grasping interface configured for removably coupling to the body in place of the first grasping interface wherein the first grasping interface is configured to mate with grasping jaws of a first surgical instrument and the second grasping interface is configured to mate with grasping jaws of a second surgical instrument having grasping jaws different in dimension or configuration from the grasping jaws of the first surgical instrument.

Some embodiments comprise an imaging device with a processing system configured to compute a current position of the imaging device within a body cavity by computing a position of a surgical instrument grasping the grasping interface by forward kinematics and determining the position of the imaging device by a known geometry of the instrument and the grasping interface.

In some embodiments the imaging device comprises a row of at least three side-facing cameras. The at least three side-facing cameras may comprise at least three pairs of the side-facing cameras wherein the at least three pairs of the side facing cameras each has a corresponding baseline distance BL, and the values of BL for the at least thee pairs of the side-facing cameras includes at least three different values for BL.

Another aspect of the invention provides a method that changes the working coordinate frame of one or more surgical instruments depending on whether images from the imaging device or images from another device are being displayed to a person controlling the surgical instruments. The coordinate frame to be used when images from the imaging device are being displayed may be determined by tracking the position and orientation of the imaging device using robotic forward kinematics.

An example method of changing working coordinate frames of other robotic arms to work in a coordinate frame at an imaging device includes computing a location of a robotic grasper, a grasping interface on the imaging device and thus the imaging units using forward kinematics of the robotic grasper and computing a transformation chain from the imaging device to the base coordinate frames of the other robotic arms and further using this transformation chain to provide comfortable control of surgical instruments carried by the other robotic arms when using views provided by the imaging device.

Another aspect of the invention provides a robotic system for minimally invasive surgery that has two simultaneous imaging devices viewing a surgical task, such that the surgeon can switch between those imaging devices in order to carry out the work in the correct frame.

Another aspect of the invention provides a robotic system for minimally invasive surgery that includes a dual surgeon console such that each surgeon controls surgical instruments while viewing images from an imaging device or images from an endoscope.

Additional aspects of the invention as well as features which maybe present in any and all combinations in different embodiments of the invention are described in the following description and/or depicted in the accompanying drawings.

### BRIEF DESCRIPTION OF THE FIGURES

The accompanying drawings illustrate one or more exemplary embodiments of the invention.

FIG. 1A is a side view showing main sections of an example imaging device.

FIG. 1B is a perspective view showing an example embodiment imaging device that includes an integrated irrigation mechanism.

FIGS. 1C and 1D are schematic cross section views showing example imaging devices having adjustable baselines for stereoscopic viewing.

FIG. 2A is a side view showing an imaging device engaged with a surgical instrument. FIG. 2B is an isometric view of the imaging device engaged with the surgical instrument. FIG. 2C is a schematic cross section of an example grasping interface.

FIG. 3 is an end view of an example imaging device showing a distal end of the imaging device orthogonal in a plane orthogonal to a longitudinal axis of the imaging device.

FIGS. 4A and 4B are respectively isometric and side elevation views of one type of surgical instrument that can be used to grasp an imaging device of the types illustrated in FIGS. 1A, 1B, 2A and 2B.

FIG. 5 is an isometric view showing an example grasping interface of an imaging device being grasped by a surgical instrument. Other parts of the imaging device are omitted for clarity. FIG. 5A is a schematic cross section view of a grasping interface of the general type shown in FIG. 5.

FIG. 6 is a schematic cross section view of a surgical field which indicates coordinate frames attached at the distal ends of two surgical instruments and an endoscope. One surgical instrument holds the proposed imaging device.

FIG. 7 is a schematic illustration of a surgical robotic setup that includes an imaging device.

### DETAILED DESCRIPTION

Detailed descriptions of one embodiment of the invention are provided herein. It is to be understood, however, that the present invention may be embodied in various forms. Therefore, the specific details disclosed herein are not to be interpreted as limiting, but rather as a representative basis for teaching one skilled in the art how to employ the present invention in virtually any detailed system, structure, or manner.

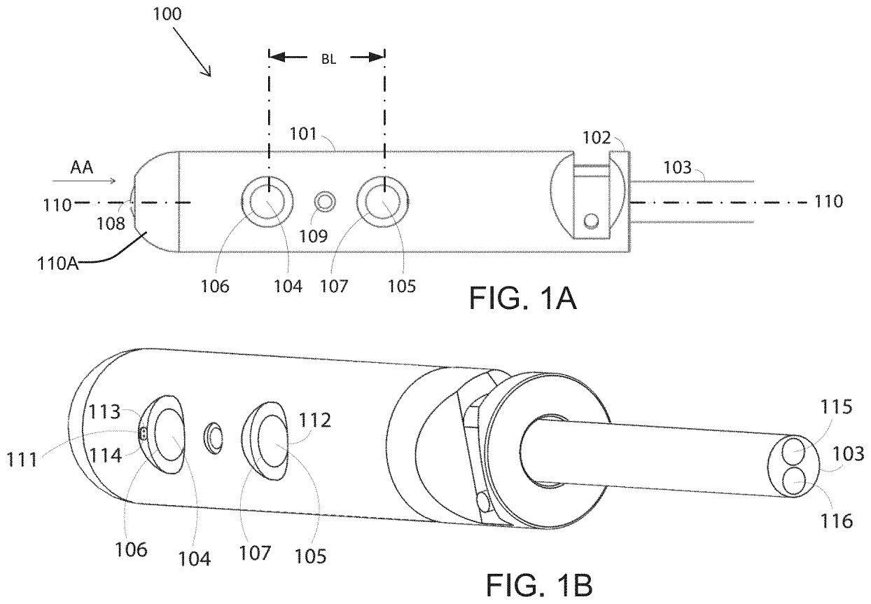

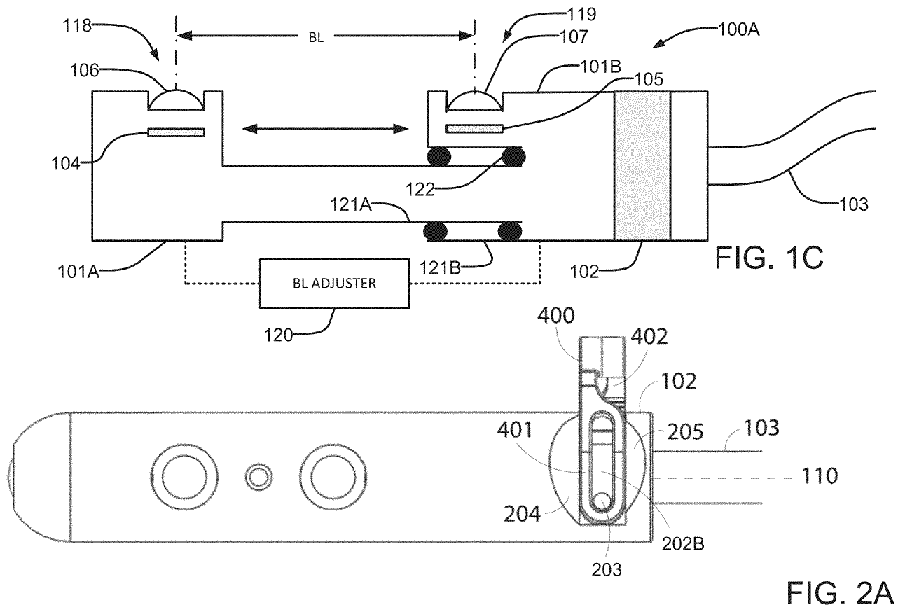

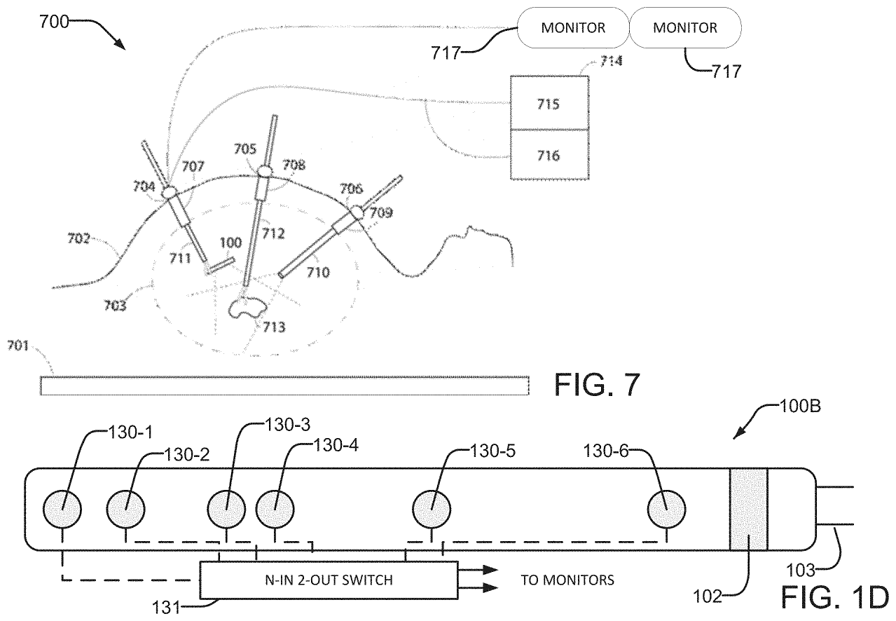

FIG. 1A is a side view an imaging device 100 according to an example embodiment of the invention. Imaging device 100 has a body 101 that is dimensioned to fit through a surgical trocar (or cannula). Body 101 preferably has a cylindrical outer surface but other geometries are possible.

Imaging device 100 includes a grasping interface 102 and is connected to a tube 103. Tube 103 may be referred to as an umbilical.

Grasping interface 102 is configured to facilitate gripping device 100 with a surgical tool. In FIG. 1A, grasping interface 102 is situated at the proximal end of the imaging device 100 (where proximal refers to the direction closer to the tube 103 and distal refers to the opposite direction). In general, grasping interface 102 can be placed anywhere along the longitudinal axis 110 of device 100.

Tube 103 may be configured to carry signals, electrical power, light and/or fluids between imaging device 100 and external support equipment. For example, tube 103 may contain cables connected to supply electrical power to imaging device 100, lumens to supply air/water for irrigating lens systems 106, 107, and/or a cable to transmit images acquired by image sensors 104, 105, 108 to the external support equipment for further processing and display. Not all of these are required. For example, in some embodiments imaging device 100 is powered by a power supply such as a battery located within main body 101 in which case a power supply cable is not required. As another example, in some embodiments device 100 includes a wireless transmitter connected to transmit images from image sensors 104, 105, 108 in which case a cable to transmit the images is not required. Where images are wirelessly transmitted from imaging device 100 the external support equipment can include a receiver configured to receive the transmitted images.

Imaging device 100 has at least two imaging sensors 104, 105. Lens systems 106, 107 are arranged to focus the light received onto the corresponding imaging sensor 104, 105. Imaging sensors 104, 105 may for example be black and white (B/W), color (e.g. red, green blue—“RGB” or red, green blue with a depth channel—“RGBD”) sensors. Imaging sensors 104, 105 may comprise any suitable technology capable of sensing images (e.g. charge coupled devices “CCD”, active pixel sensors “APS” etc.).

Lens systems 106, 107 are arranged to collect light at locations that are spaced apart from one another along body 101 by a distance BL. Lens systems 106, 107 collect light from overlapping fields of view. Preferably, lens systems 106, 107 have optical axes that are parallel to one another.

Lens systems 106, 107 and corresponding imaging sensors 104, 105 may be operated to obtain stereoscopic views of areas inside of a body cavity and to provide the stereoscopic views to a surgeon. The surgeon may view the stereoscopic views in a way that provides depth perception. For example, the surgeon may:
view the stereoscopic images while the stereoscopic images are displayed on two external monitors, each of which is presented to a corresponding eye of the surgeon through a viewfinder (left monitor to the left eye, and right monitor to the right eye). This is the approach taken for displaying stereoscopic images in the da Vinci™ surgical system from Intuitive Surgical.
view the stereoscopic images presented on a single display in a way that allows a left eye image to be directed to the surgeon's left eye and the right eye image to be directed to the surgeon's right eye. For example, left and right images may be displayed in a time multiplexed manner and optical shutters may be arranged to block the right-eye image from the surgeon's left eye when the right eye image is being displayed and to block the left-eye image from the surgeons right eye when the left-eye image is being displayed. As another example, left- and right-eye images may be displayed with light that is separable. For example, the left- and right-eye images have different polarizations or may be composed of primary colors that have different spectra. Polarization filters or spectral filters may be provided to block the left-eye images from reaching the surgeon's right eye and to block the right-eye images from reaching the surgeon's left eye.
view the stereoscopic images presented on a holographic display.
view the left and right eye images on a 3D display.
wear a virtual reality (VR) headset.
use any of the wide range of 3D viewing technologies that are commercially available and/or described in the patent literature and/or described in the technical literature.
   Any of these approaches allow the surgeon to view stereoscopic views of a location of a surgical procedure while controlling a robotic system to perform the surgical procedure.

The distance BL is called the baseline. Baseline BL may be at least 5 mm (the standard baseline between the two imaging sensors of a typical surgical endoscope). Preferably BL is longer than 5 mm. Because les systems 106, 107 are oriented to take in light from the side of body 101 the diameter of a trocar or other lumen through which imaging device 100 is introduced does not impose any limits on the length of baseline BL. For example, baseline BL may have a length in the range of 5 mm to 45 mm (e.g. 5 mm, 7 mm, 10 mm 15 mm, 20 mm, 29 mm, 32 mm etc.).

In some embodiments baseline BL is adjustable. FIG. 1C shows an example imaging device 100A in which the distance BL between the camera 118 provided by imaging sensor 104 and lens system 106 and the camera 119 provided by imaging sensor 105 and lens system 107 is adjustable. Otherwise imaging device 100A can have features like any other feature or combination of features of any other imaging device described herein. In imaging device 100A, cameras 118 and 119 are in parts 101A and 101B of housing 100 that are longitudinally movable relative to one another. For example one of parts 101A and 101B may telescope within the other one of parts 101B and 101A or one of parts 101A and 101B may be mounted to slide along a member that is connected to the other one of parts 101B and 101A or parts 101A and 101B may be coupled by a variable length mechanical linkage etc.

In FIG. 1C, male part 121A of part 101A is telescopically received in female part 121B of part 101B. Seals 122 are schematically illustrated.

A BL adjuster 120 allows adjustment of distance BL. BL adjuster 120 may comprise any of:
a simple mechanism which allows BL to be adjusted before imaging device 101A is put to use. For example: a screw that may be turned manually to adjust BL by moving housing part 101A relative to housing part 101B, a locking member such as a screw or pin that may be loosened or removed to allow housing part 101A to be slid to a desired position relative to housing part 101A and then replaced; a detent mechanism that holds housing part 101A relative to housing part 101B at any of a plurality of positions and allows a user to push or pull housing part 101A until it is in a desired one of the detented positions; and so on.
a mechanical mechanism that can be set using surgical instruments while imaging device 100A is in a body cavity. An example of such a mechanism is a spring loaded detent mechanism configured to allow BL to be adjusted by pushing part 101A toward part 101B until a desired detent position is reached. In some embodiments the detent mechanism will release to allow a spring to push part 101A to a fully extended position when part 101A has been pushed fully toward part 101B.
a power actuator such as a powered lead screw, pneumatic or hydraulic actuator controlled from outside of the body and connected to move part 101A relative to part 101B continuously and/or in steps.

Another way to permit adjustment of BL is to provide an imaging device that includes three or more side-facing cameras spaced apart along body 101. In some embodiments optical axes of adjacent ones of the three or more cameras are spaced apart along body 101 by different distances. For example, three cameras spaced apart along body 101 such that first and second cameras are spaced apart by 5 mm and the second camera is spaced apart from a third camera by a distance of 10 mm can be taken in any of three pairs to provide BL of 5 mm (first and second cameras), 10 mm (second and third cameras) and 15 mm (first and third cameras). Providing a fourth camera can provide more values of BL to select among. For example, a fourth camera may be spaced apart along body 101 from the third camera by a distance of 20 mm to provide baselines of 20 mm (third and fourth cameras), 30 mm (second and fourth cameras) and 35 mm (first and fourth cameras). These particular spacings are only non-limiting examples. In some embodiments a row of three or more cameras may be equally spaced apart from one another along body 101. In some embodiments cameras are spaced apart from one another in a row of cameras by a distance in the range of 4 mm to 12 mm.

In embodiments which provide a row of three or more cameras, a switching circuit may select a baseline by taking images from a selected pair of the three or more cameras. The switching circuit may be included in an imaging device as described herein or may be part of an external system. FIG. 1D shows schematically an example imaging device 100B that includes a row of six side-facing cameras 130-1 to 130-6 (collectively or generally cameras 130). A switch 131 selects images from two of cameras 130 which have optical axes spaced apart by a desires baseline distance. Otherwise imaging device 100B can have features like any other feature or combination of features of any other imaging device described herein.

An imaging device that provides an adjustable value of BL can be applied to optimize stereo disparity as a function of the distance between an imaging device and the object/task that is being observed/performed. When the distance to disparity ratio is very large, it is difficult to perceive depth accurately because of the small triangulation angle. When the distance to disparity ratio is too small, the object/task are observed/performed “cross-eyed”.

An experimental user study conducted using multiple imaging devices constructed to provide different values for BL found that for a distance of 20 cm between the imaging device and a site being observed a BL longer than 5 mm (which is the current endoscopic baseline) improved the depth perception of subjects and aided in greater performance of the task. The values of BL tested were 10 mm, 15 mm, 20 mm, and 30 mm. Results of this study showed an improvement in task performance as BL was increased with peak performance when BL was 20 mm.

Imaging device 100 preferably includes at least one illumination source 109 which is operable to emit illumination. Illumination source 109 may, for example, comprise one or more light emitting diode (LED), a laser, or a structured light system. Illumination source 109 may emit light of any wavelength or wavelength or spectral composition that may be detected by imaging sensors 104, 105 (any wavelength (e.g. visible, infrared, and/or ultraviolet spectrum).

Example imaging device 100 has two side-facing imaging sensors 104, 105, one end-facing imaging sensor 108 and one source of illumination 109. Other embodiments may include additional sensors and/or additional sources of illumination.

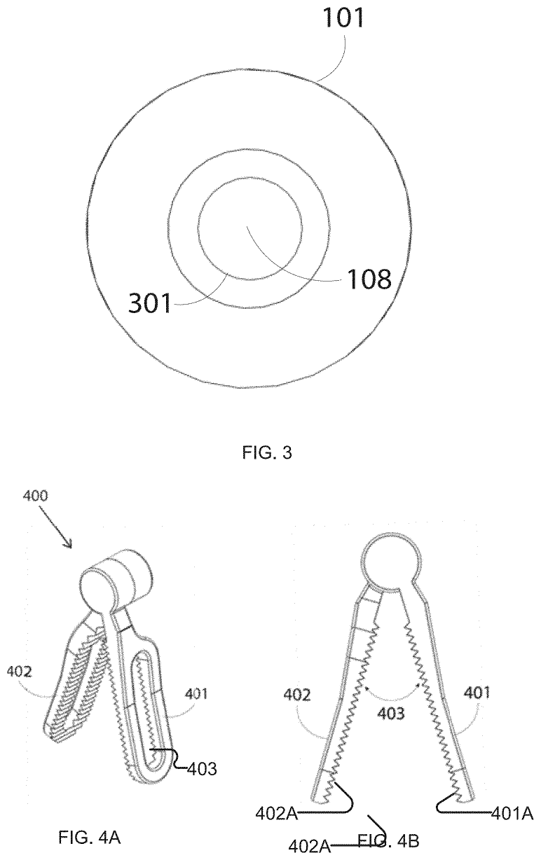

FIG. 3 shows an example configuration for the distal end of imaging device 100 taken along direction AA (FIG. 1A) orthogonal to axis 110. In this example embodiment, at least one imaging sensor 108 is present on the distal end 110A of imaging device 100. One or more sources of illumination may be present on end 110A. Image sensor 108 includes a lens system 301 arranged to focus light onto sensor 108. Images from sensor 108 may be used to guide the insertion of imaging device 100 (e.g. when it is first inserted into a body cavity through a surgical trocar).

FIG. 1B illustrates an example irrigation mechanism that may be provided to clean outer surfaces of lens systems 106, 107. During surgical procedures, there is a tendency for outer surfaces of imaging devices to become soiled with blood and other bodily fluids. Openings 111 and 112 are arranged to provide irrigation for lens systems 106, 107. Opening 111 has orifices 113, 114 connected to supply water and air respectively. Water is sprayed first through orifice 113 to clean the outer surface of lens system 106 of any bodily fluids that may be present. Pressurized air is then released through orifice 114 to dry any remaining water droplets. Opening 112 may also have orifices connected to deliver air and water (although such orifices cannot be seen in FIG. 1A). Openings 111, 112 are internally connected to conduits which are respectively connected to carry water and air to orifices 113, 114. These conduits may be provided by lumens within tube 103 or by separate tubes for the supply of water and air. Irrigation may be performed with a single conduit by first delivering water through the conduit and then delivering air through the conduit.

Providing a mechanism for cleaning outer surfaces of lens systems 106, 107 without any need to remove imaging device from the body cavity for cleaning can allow a surgeon to clean the optics of the imaging device more frequently and can reduce the time taken to perform each cleaning.

FIG. 1A shows a cut through tube 103. In this embodiment tube 103 encloses power supply cable 115 and an irrigation supply cable 116. Cable 115 may be used to supply power as well as transmit the images received by sensors 104, 105, 108. Other embodiments of the device can include separate cables for the power supply and transmission of images as desired. The proximal end of irrigation supply tube 116 (not shown) is connectable to an external supply of water and pressurized air (not shown).

Grasping interface 102 is designed to be compatible with a suitable surgical instrument and this allows for repeatable grasping of imaging device 100 whenever necessary. The surgical instrument may be used to position and securely hold imaging device 100 in a desired position to provide clear images of a site of interest.

The full range of motion allowed by the surgical instrument may be used to position and orient imaging device 100. This may facilitate a very wide range of surgical viewing directions and allows adjustment of the viewing direction through more degrees of freedom.

Grasping interface 102 enables fast and easy pick-up and release of an imaging device 100. In some embodiments, grasping interface 102 is designed to be used with a surgical instrument such as the one illustrated in FIG. 4A and FIG. 4B. Grasping interface 102 allows a suitable surgical robotic or laparoscopic instrument to grasp and manipulate imaging device 100. The suitable surgical instrument 400 shown in FIGS. 4A and 4B has first and second grasping jaws 401, 402 situated in an opposing relationship, with a variable grasping angle 403 between grasping jaws 401, 402. The surgical instrument illustrated in FIG. 4 is similar to the ProGrasp™ instrument (Intuitive Surgical, CA). The example grasping interface 102 shown in FIG. 1B has been designed to mate with the jaws 401, 402 of this instrument. Advantageously, a grasping interface 102 may be dimensioned and configured for use with any of a wide range of clinically approved surgical instruments.

Preferably grasping interface 102 engages with the tips of jaws 401, 402 so that no part of instrument 400 protrudes past imaging device 100. Projecting jaw tips would be undesirable because they could be in the way of other instruments and/or could undesirably contact tissues in the body cavity while instrument 100 is being held. This is not mandatory however.

In some embodiments imaging device 100 has a detachable and interchangeable grasping interfaces 102. Different grasping interfaces may be designed to be engaged by different surgical instruments. This facilities the use of two, more than two or many different designs of grasping interface 102 to enable the use of different surgical instruments to grasp an imaging device 100. Such a design also introduces more flexibility in the compatibility of imaging devices 100 with different currently existing or future developed surgical robotic systems and tools.

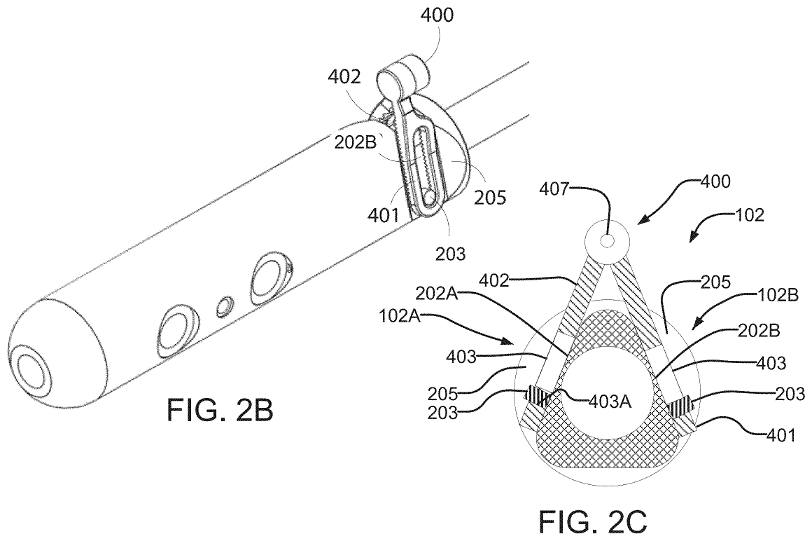

FIG. 2A and FIG. 2B illustrate an example embodiment of a grasping interface 102 in more detail. FIG. 2C is a schematic cross section of an example grasping interface 102. In the illustrated embodiment, grasping interface 102 includes opposing grooves 102A, 102B each dimensioned to receive one jaw 401, 402 of a tool 400. Sides 204, 205 of grooves 102A, 102B are tapered. Bottom surfaces 202A, 202B of grooves 102A and 102B are angled to match the angle of grasping surfaces 401A, 402A of jaws 401, 402. Grasping interface 102 may include projections 203 such as posts that are received in recesses or openings such as slots 403 in the jaws of instrument 400 when instrument 400 is grasping interface 102.

Bottom surfaces 202A and 202B may have widths that match widths of jaws 401, 402 of instrument 400 such that when instrument 400 grasps grasping interface 102 the angle of instrument 400 and imaging device 100 is fixed and repeatable. Grooves 102A and 102B may be oriented such that when they are engaged by instrument 400, instrument 400 is orthogonal to the longitudinal axis 110 of imaging device 100.

To grasp imaging device 100, the surgeon controls an instrument 400 to approach grasping interface 102 such that jaws 401, 402 of the instrument are opened to a large (or maximum) angle. The jaws 401, 402 are then positioned over the surfaces 202A and 202B. During this approach, instrument 400 may be in an ‘unlocked’ position. Closing jaws 401, 402 brings the inner surfaces 401A, 401B of the jaws in contact with the corresponding surfaces 202A, 202B on grasping interface 102 of imaging device 100 and thus encloses the imaging device 100 within the jaws 401, 402 of the surgical instrument 400.

Because base surfaces 202A, 202B form a wedge, clamping jaws 401, 402 against base surfaces 202A, 202B can tend to force grasping interface 102 away from instrument 400 as indicated by arrow 211. This action tends to engage projections 203 against ends 403A of slots 403.

In the example embodiment shown in FIG. 2C and some other embodiments the interaction of base surfaces 202A, 202B and projections 203 with instrument 400 repeatably positions grasping interface 102 relative to implement 400 as jaws 401, 402 of implement 400 are closed onto grasping interface 102. The distance between pivot axis 407 and grasping interface 102 is set by the engagement of projections 203 with the ends of slots 403. The angle of jaws 401, 402 relative to longitudinal axis 110 of device 100 is set by the engagement of jaws 401, 402 between the bases of sloping sidewalls 204, 205. Base surfaces 202A, 202B are angled at an angle that matches the angles of jaws 401, 402 when instrument 400 is closed on grasping interface 102.

FIG. 2A and FIG. 2B show surgical instrument in 400 in a ‘locked’ position i.e., engaged with the grasping interface 102. The manner in which instrument 400 moves from the ‘unlocked’ to the ‘locked’ position may be similar to that described in Schneider et al., patent publication US20130338505A1 titled ‘Ultrasound probe for laparoscopy’, the entirety of which is hereby incorporated herein by reference.

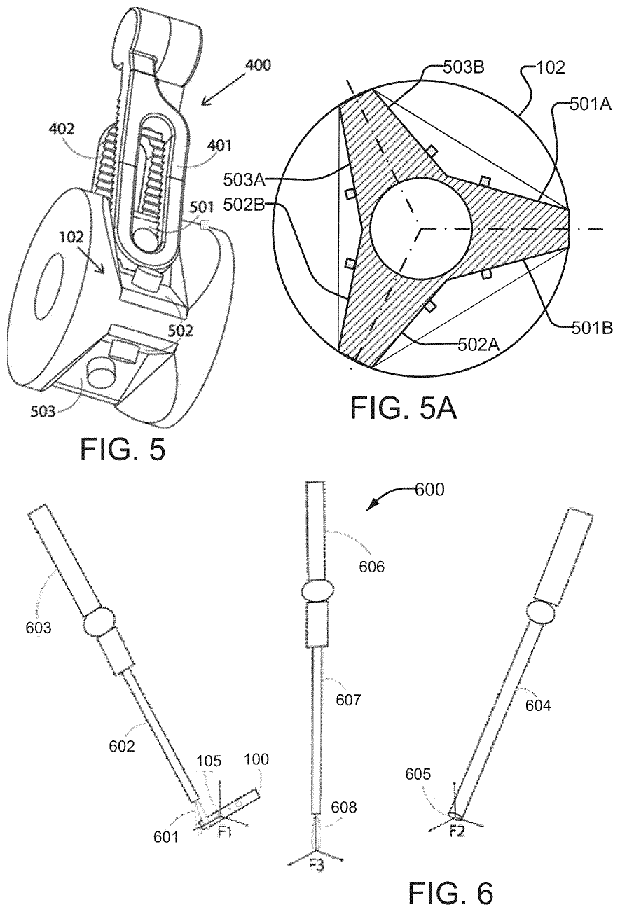

In some embodiments, grasping interface 102 has more than one set of graspable surfaces. FIG. 5 is an example embodiment of a grasping interface 102 that provides three such sets of graspable surfaces 501, 502, 503. FIG. 5A is a schematic cross section view of a grasping interface of the type shown in FIG. 5. FIG. 5A shows that grasping surfaces 501A and 501B make up one set 501 of grasping surfaces, grasping surfaces 502A and 502B make up a second set 502 of grasping surfaces and grasping surfaces 503A and 503B make up a third set 503 of grasping surfaces. Each set of grasping surfaces may be symmetrical about a plane that includes a longitudinal axis 110 of an imaging device (such planes are indicated by dash-dot lines in FIG. 5A). Each set of grasping surfaces may have features of construction and may be used as described elsewhere herein (for example with reference to FIGS. 2A, 2B and 2C).

A surgeon can grasp the imaging device 100 through such a grasping interface 102 such that instrument jaws 401, 402 close over any one of the sets of surfaces 501, 502 or 503 in a manner similar to that described above. The presence of more than one set of graspable surfaces makes it easier to quickly and repeatably grasp imaging device 100. Providing plural sets of grasping surfaces as shown, for example in FIGS. 5 and 5A enables the surgeon to pick up the imaging device 100 regardless of what the camera angle is relative to the surface it rests on (angle of the cable 103 relative to instrument 400). This can reduce the time taken to reposition imaging device 100 within the body cavity such that the jaws 401, 402 can be suitably positioned over the grasping interface 102 to facilitate smooth and easy grasping.

FIG. 5 is merely one example of a construction which provides plural sets of graspable surfaces. A grasping interface 102 of an imaging device 100 may include one, two, three or more sets of graspable surfaces.

Grasping interface 102 can advantageously be designed so that a surgical instrument 400 grasping a particular set of grasping surfaces always holds imaging device 100 in the same position and orientation relative to instrument 400.

In conventional robot-assisted minimally invasive surgical systems, a surgeon teleoperatively controls robotic arms at the patient-side by operating master controllers present at a surgical console. At the patient-side, an endoscopic camera provides images of the surgical field. One can define a coordinate frame at the endoscope, with the Z axis pointing into the view and the Y axis pointing upwards in the view provided by the endoscope. To facilitate visual-motor consistency between the movements of the surgeon's master controllers and the movements of the patient-side robotic arms as seen through the endoscope, the tips of the surgical instruments attached to the robotic arms move with respect to the coordinate system at the endoscope. Such a system ensures intuitive control of surgical instruments.

When using an imaging device 100 as described herein in addition to or instead of a standard endoscope it is desirable to provide a way to facilitate visual-motor alignment when working with images from the imaging device 100. Without such visual-motor alignment the surgeon may experience mental and physical strain.

FIG. 6 schematically shows a system 600 according to one embodiment of the invention. A coordinate frame F1 is defined at imaging sensor 105 of imaging device 100. It may be convenient to make the Z axis point into the view and the Y axis point upward in the view provided by imaging device 100. Imaging device 100 is grasped by the tip 601 of a surgical instrument 602 which in turn is carried by a robotic arm 603.

System 600 also comprises an endoscope 604 with coordinate frame F2 defined at its tip 605. Another coordinate frame F3 is defined at a tip 608 of a surgical instrument 607 carried by another robotic arm 606.

A method according to the invention comprises changing the base working frame of surgical instrument 607 from coordinate frame F2 to coordinate frame F1 when switching to view images from the imaging device 100. The method may change the base working frame of surgical instrument 607 from coordinate frame F1 to coordinate frame F2 when switching back to view images from endoscope 604. A console may comprise an image selector control which may be used to select a source of images (e.g. endoscope 605 or imaging device 100. Switching the working coordinate frame for instrument 607 may be triggered by operation of the image selector control.

The locations (position and orientation) of instrument tips 601 and 608 inside the body cavity can be computed through forward kinematics of robotic arms 603 and 606. For example, robotic arms 603 and 606 may have segments of a known geometry connected by joints. The position of an instrument tip 601 carried by a robotic arm may therefore be computed based on the known geometry of the robotic arm and the instrument as well as known configuration of the joints of the robotic arm. For example, the robotic arm may include sensors that report on the positions of the joints.

The position and orientation of imaging device 100 is predefined and known with respect to the grasping interface 102 and thus the instrument tip 601 because of the unique mating between the grasping interface 102 and the jaws of the grasping instrument 602. The transformation from tool tip 607 to the coordinate frame F1 present at the imaging sensor 105 is then computed (e.g. by forward kinematics) and used to establish visual-motor consistency.

Optionally, the relative positions of an imaging device 100 and surgical instruments or other features relevant to the surgical task being performed may be determined in other manners such as using external sensors and external tracking systems; external calibration objects and processing of images taken by the imaging device (computer vision methods). The inventors consider that the reverse kinematic method described above is preferable to such other methods at least in part because forward kinematics is not difficult to implement and is reliable.

The orientation of imaging device 100 may be selected to satisfy varying ergonomic factors. For example, for bi-manual tasks (where a user controls two surgical instruments), it could be beneficial to position imaging device 100 so that it is aligned with an axis that is an average of axes of left and right instruments. As another example, where there is a prevailing normal direction to a region of tissue relevant to the surgical tasks being carried out, it may be beneficial to orient imaging device 100 to be perpendicular to this prevailing normal direction.

In some embodiments an imaging device 100 and an endoscope 604 are both used in robot-assisted minimally invasive surgical procedures. Imaging device 100 can be placed at any point within the body cavity; its position is only limited by the point of insertion of the robotic arm holding it and the capabilities of the robotic arm. Example positions include directly opposite to endoscope 604, or at an angle of 90 degrees (about the vertical axis) to endoscope 604.

FIG. 7 schematically illustrates a system 700 for robot-assisted minimally invasive surgery which includes an imaging device 100. A patient 702 lies on the patient-side bed 701 for the procedure. The patient's abdomen is insufflated with a gas (usually carbon dioxide) to provide more space within the abdominal cavity 703 for the instruments to be inserted. Three incisions 704, 705, 706 are made through which surgical trocars 707, 708, 709 are inserted. Endoscope 710 and surgical instruments 711, 712 are each inserted through a corresponding one of trocars 707, 708, 709. Surgical instrument 712 holds imaging device 100 by grasping interface 102. Surgical instrument 711 is operating on tissue 713. Tube 103 connects imaging device 100 to an external system 714 for power supply 715 and irrigation 716 and also to the surgeon's monitors 717.

A surgeon can use both endoscope 710 and imaging device 100 to view images of tissue 713. The visual-alignment method described above may be performed to help the surgeon to easily control the robotic system to complete the surgical task even when switching back and forth between imaging device 100 and endoscope 710.

In some example embodiments an imaging device is built into a surgical instrument. In such embodiments grasping interface 102 is replaced by a wrist of an instrument configured to be carried by a robotic arm. The imaging device may be like imaging device 100 in other respects. The imaging device may be positioned in a body cavity by moving the robotic arm and moving the wrist. The wrist may be positioned so that the imaging device is aligned with the rest of the instrument while introducing the imaging device through a trocar or other lumen. Once inside the body cavity the wrist may be flexed to obtain a desired view of a surgical site. Such a ‘wristed-camera instrument’ may be carried by a robotic arm of a minimally invasive surgical robot similar to existing surgical instruments like the ProGrasp™ (Intuitive Surgical, CA). Control of such a wristed-camera instrument may be performed using master controllers at the surgeon console. Actuation of the wrist may be provided in the same manner as actuation of other surgical instruments for use with a patient-side robot.

The visual-alignment method described above may be applied when a wristed-camera instrument is being used. A wristed-camera instrument can be used along with another imaging system such as an endoscope to provide plural views of the surgical scene.

In yet another example embodiment, a wristed-camera instrument or an imaging device 100 can be employed in a dual-console minimally invasive surgical robotics system. Such a system has two surgeon consoles that control a single patient-side robot. One surgeon may control surgical instruments with respect to images from an endoscope while the other surgeon may control surgical instruments with respect to images from the wristed-camera instrument or the imaging device 100. In another example embodiment, one surgeon may control surgical instruments with respect to images from a first wristed-camera instrument or imaging device 100 while the other surgeon may control surgical instruments with respect to images from a second wristed-camera instrument or imaging device 100.

It can be appreciated that imaging devices according to a wide range of embodiments of the invention may be designed to provide small imaging devices that can be inserted through a surgical trocar and grasped by a surgical instrument. For example, such imaging devices may be designed to fit through trocars that have inside diameters of 11, 6 or 3.5 mm. For example, such imaging devices may have cylindrical bodies that are only slightly less than the inside diameter of a trocar with which they are intended to be used. Such imaging devices may be small enough to be easily inserted through existing trocars or natural body orifices without needing to modify the size of the incision on the body. At the same time, such imaging devices may provide relatively long baselines for stereo vision.

As mentioned above, some embodiments provide an illuminator which emits structured light. The structured light could be in the infrared (IR) wavelength, which is invisible to the surgeon and thereby does not hinder sight of the surgical scene. Structured light may be captured by imaging units (e.g. 104, 105) and processed to capture depth information. The depth information may be provided to a surgeon. In some embodiments the structured light comprises a pattern of stripes or a grid.

Imaging devices as described herein may be positioned using a surgical instrument carried by a robotic arm or by an integrated surgical instrument. In either case, control of the movement, position and pose of the imaging device is through control of the surgical instrument itself. The surgical instrument may be controlled using a suitable control console. This eliminates the need for additional non-standard control hardware and does not disrupt the surgical workflow during the surgery.

### Interpretation of Terms

Unless the context clearly requires otherwise, throughout the description and the
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Methods of the invention (e.g. for adjusting base coordinate systems used to control a surgical robot) may be implemented in whole or part using specifically designed hardware, configurable hardware, programmable data processors configured by the provision of software (which may optionally comprise “firmware”) capable of executing on the data processors, special purpose computers or data processors that are specifically programmed, configured, or constructed to perform one or more steps in a method as explained in detail herein and/or combinations of two or more of these. Examples of specifically designed hardware are: logic circuits, application-specific integrated circuits (“ASICs”), large scale integrated circuits (“LSIs”), very large scale integrated circuits (“VLSIs”), and the like. Examples of configurable hardware are: one or more programmable logic devices such as programmable array logic (“PALs”), programmable logic arrays (“PLAs”), and field programmable gate arrays (“FPGAs”). Examples of programmable data processors are: microprocessors, digital signal processors (“DSPs”), embedded processors, graphics processors, math co-processors, general purpose computers, server computers, cloud computers, mainframe computers, computer workstations, and the like. For example, one or more data processors in a control system for a robotic surgery system may implement methods as described herein (e.g. to switch between reference base coordinate frames) by executing software instructions in a program memory accessible to the processors.

Certain aspects of the invention may also be provided in the form of a program product. The program product may comprise any non-transitory medium which carries a set of computer-readable instructions which, when executed by a data processor, cause the data processor to execute a method of the invention. Program products according to the invention may be in any of a wide variety of forms. The program product may comprise, for example, non-transitory media such as magnetic data storage media including floppy diskettes, hard disk drives, optical data storage media including CD ROMs, DVDs, electronic data storage media including ROMs, flash RAM, EPROMs, hardwired or preprogrammed chips (e.g., EEPROM semiconductor chips), nanotechnology memory, or the like. The computer-readable signals on the program product may optionally be compressed or encrypted.

Where a component (e.g. a surgical implement, lens system, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described above. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

It is therefore intended that the following appended claims and claims hereafter introduced are interpreted to include all such modifications, permutations, additions, omissions, and sub-combinations as may reasonably be inferred. The scope of the claims should not be limited by the preferred embodiments set forth in the examples, but should be given the broadest interpretation consistent with the description as a whole.

1. An imaging device comprising:
an elongated body;
a plurality of side-facing cameras each comprising an imaging sensor and a lens system, the cameras spaced apart from one another in a direction along the body and facing sideways; and
a grasping interface on the body, the grasping interface configured to be grasped by jaws of a surgical instrument.

2. The imaging device according to claim 1 wherein the grasping interface comprises first and second opposed grooves extending transversely to the body, the first and second opposed grooves comprising inclined sidewalls such that a base surface of the first groove is narrower than an opening of the first groove and a base surface of the second groove is narrower than an opening of the second groove.

3. The imaging device according to claim 2 wherein the base surface of the first groove is inclined at an acute angle relative to the base surface of the second groove.

4. The imaging device according to claim 3 comprising first and second projections extending generally perpendicularly from the base surfaces of the first and second grooves respectively.

5. The imaging device according to claim 4 wherein the first and second projections are spaced apart from the sidewalls of the first and second grooves respectively.

6. The imaging device according to claim 1 wherein the grasping interface comprises a plurality of sets of base surfaces, each of the sets of base surfaces comprising a first base surface inclined at an acute angle to a second base surface.

7. The imaging device of claim 6 wherein each of the pairs of base surfaces is symmetrical about a plane that includes a longitudinal axis of the body and the pairs of base surfaces are equally angularly spaced apart around a circumference of the body.

8. The imaging device according to claim 7 comprising first and second projections extending generally perpendicularly from the first and second base surfaces of each of the pairs of base surfaces.

9. The imaging device according to claim 7 wherein the grasping interface comprises three to five pairs of the base surfaces.

10. The imaging device according to claim 1 wherein the body is cylindrical and has an outside diameter of less than 11 mm.

11. The imaging device according to claim 2 comprising an additional camera comprising an imaging sensor and a lens system on a distal end of the body.

12. The imaging device according to claim 1 comprising an umbilical connected to a proximal end of the body.

13. The imaging device according to claim 12 wherein the umbilical comprises at least one lumen in fluid communication with one or more orifices, the one or more orifices located adjacent to and oriented to eject fluid onto an outer surface of the lens system of at least one of the cameras.

14. The imaging device according to claim 1 wherein a baseline distance BL between optical axes of first and second ones of the plurality of side-looking cameras is adjustable.

15. The imaging device according to claim 14 wherein the elongated body comprises a first part carrying the first one of the plurality of side-looking cameras and a second part carrying the second one of the plurality of side-looking cameras and the first part is telescopically received in the second part.

16. The imaging device according to claim 14 comprising a power actuator connected to drive the first one of the plurality of side-looking cameras toward or away from the second one of the plurality of side-looking cameras.

17. The imaging device according to claim 1 wherein the grasping interface is provided by a first grasping interface that is detachably coupled to the body and the imaging device comprises a second grasping interface configured for removably coupling to the body in place of the first grasping interface wherein the first grasping interface is configured to mate with grasping jaws of a first surgical instrument and the second grasping interface is configured to mate with grasping jaws of a second surgical instrument having grasping jaws different in dimension or configuration from the grasping jaws of the first surgical instrument.

18. The imaging device according to claim 1 in combination with a processing system configured to compute a current position of the imaging device within a body cavity by computing a position of a surgical instrument grasping the grasping interface by forward kinematics and determining the position of the imaging device by a known geometry of the instrument and the grasping interface.

19. The imaging device according to claim 1 wherein the plurality of side-facing cameras comprises a row of at least three side-facing cameras.

20. The imaging device according to claim 1 wherein the at least three side-facing cameras comprises at least three pairs of the side-facing cameras, the at least three pairs of the side facing cameras each has a corresponding baseline distance BL, and the values of BL for the at least thee pairs of the side-facing cameras includes at least three different values for BL.
